(19) United States
(12) Patent Application Publication
(10) Pub. No.: US 2020/0054233 A1
(43) Pub. Date: Feb. 20, 2020

(54) METHOD FOR DETERMINING A PLURALITY OF ACTION POTENTIALS IN THE HEART

(71) Applicant: BIOTRONIK SE & Co. KG, Berlin (DE)

(72) Inventor: Ovidiu CODREANU, Berlin (DE)

(21) Appl. No.: 16/498,583

(22) PCT Filed: Mar. 29, 2018
(86) PCT No.: PCT/EP2018/058051

§ 371 Date: Sep. 27, 2019

(30) Foreign Application Priority Data
Apr. 3, 2017 (DE) 10 2017 107 082.6

(51) Int. Cl. A61B 5/0468; A61B 5/0408; A61B 5/042; A61B 5/00; A61B 5/044; A61B 5/0432

(52) U.S. Cl. CPC A61B 5/0468; A61B 5/04085; A61B 5/042; A61B 5/0432; A61B 5/7455; A61B 5/044; A61B 5/7405

(57) ABSTRACT

The disclosure relates to a method for determining a plurality of action potentials in the heart having the following steps:

a) Recording a surface ECG signal synchronously with at least 64 channels,

b) Recording at least one IEGM signal,

c) Processing the surface ECG signal by means of ICA analysis and determining the sum and position of a plurality of action potentials in the heart based on the ICA analysis, and,

d) Comparing the at least one IEGM signal to the plurality of action potentials and correcting the sum and/or position of at least one of the plurality of action potentials in the heart based on this comparison.

The disclosure further relates to a corresponding device, a corresponding computer program product, and a corresponding system.

### CROSS-REFERENCE TO RELATED APPLICATIONS

This patent application is the United States national phase under 35 U.S.C. § 371 of PCT International Patent Application No. PCT/EP2018/058051 filed on Mar. 29, 2018, which claims the benefit of German Patent Application No. DE 10 2017 107 082.6, filed on Apr. 3, 2017, the disclosures of which are hereby incorporated by reference in their entireties.

### TECHNICAL FIELD

The present invention describes a method for determining a plurality of action potentials in the heart, a corresponding computer program product, a corresponding device, and a corresponding system.

### BACKGROUND

One known method for non-medication-based, minimally invasive treatment of idiopathic or paroxysmal arrhythmias of the heart is intracardiac ablation. In this case, for instance when there is atrial fibrillation, a catheter having at least one electrode is inserted via the venous blood vessels into the right atrium of the heart and guided through the cardiac septum into the left atrium. Most ablation catheters have 4 electrodes, HF energy being emitted to the tissue via the electrode at the tip of the catheter. Then muscle tissue areas in the left atrium are destroyed (ablated) by means of a high frequency current introduced by the electrode such that so-called rotors (circling excitations) or ectopic action sources that are considered causal for the atrial fibrillations are eliminated. Alternatively, an analogous minimally invasive treatment by means of laser or cold may be undertaken.

It is possible to increase the success rate of an ablation if the effectiveness of the lesions induced by the ablation or other minimally invasive methods can be assessed more reliably during the treatment. This is only possible to a limited extent with the current methods. In many cases, therefore, a second atrial fibrillation (AF) ablation treatment is necessary after a first AF ablation. This leads to increased stress on the patient.

Providing a surface electrocardiograph (ECG), i.e., a recording of the electrical activity of cardiac muscle fibers by means of electrodes applied to the surface of the patient's body, has been an option for detecting cardiac arrhythmias for many years. Surface ECGs are also already used for evaluating the outcome of an ablation.

Methods are known from U.S. Pat. No. 7,123,954 B2 that evaluate surface ECG signals and intracardiac ECG signals of an electrode arranged in the heart by means of a so-called temporospatial morphology correlation in order to be better able to evaluate arrhythmias, to better identify possible ablation sites, and to assess the success of ablation. Intracardiac excitation (pacing) is conducted for recording the intracardiac ECG signals. The reproducibility of a time-dependent and electrode-specific electrograph morphology is analyzed in the temporospatial morphology correlation.

U.S. Pat. No. 9,370,312 B2 describes different calculation methods for comparing a surface ECG to an intracardiac ECG by means of maps of electrical potentials of the heart. An endocardial map is produced by means of catheter and a map is produced based on a surface ECG and the maps are correlated using fixed anatomical points and/or electrical properties of the maps.

US 2012/0232417 A1 discloses a method for analyzing signals for determining a condition of a heart.

The article entitled “Noninvasive Study of the Human Heart using Independent Component Analysis,” Yi Zhu et al., Proceedings of Sixth IEEE Symposium on BioInformatics and BioEngineering (BIBE'06) (2006), page 340, describes the use of Independent Component Analysis (ICA) on ECG data for simulating cardiac activity.

The document entitled “Predicting Catheter Ablation Outcome in Persistent Atrial Fibrillation Using Atrial Dominant Frequency and Related Spectral Features,” M. Garibaldi et al., Engineering in Medicine and Biology Society, 34th Annual International Conference of the IEEE EMBS (2012), pages 613-616, describes a method for determining outcome of an ablation.

The methods described in the foregoing frequently have the drawback that they still work too imprecisely and in some cases can only demonstrate an interruption in the signal path in the surface of the cardiac muscle due to ablation, but cannot detect signal paths deep in the cardiac muscle. Therefore the outcome of an ablation is frequently not assessed correctly.

The present invention is directed toward overcoming one or more of the above-mentioned problems.

### SUMMARY

An object may be considered to be a method that permits more precise assessment of ablation, including areas disposed deeper. An object furthermore is to provide a corresponding device.

A method having the features of claim 1, a computer program product according to claim 8, a device according to claim 9, and a system according to claim 15 are provided. Additional embodiments are the subject matter of dependent claims.

In particular, the magnitude and arrangement of a plurality of action potentials in the heart may be determined with the steps listed as follows:

a) Synchronously recording at least one surface ECG signal with a plurality of channels,

b) Recording at least one IEGM signal,

c) Processing the surface ECG signal by means of ICA analysis and determining the sum and position of a plurality of action potentials in the heart based on the ICA analysis, and,

d) Comparing the at least one IEGM signal to the plurality of action potentials and correcting the sum and/or position of at least one of the plurality of action potentials in the heart based on this comparison.

It may be provided that the recording of the surface ECG signal takes place synchronously for all channels (e.g. 64 channels). Moreover, the synchronous recording of the surface ECG signal may take place synchronously with the recording of the IEGM signal. The synchronicity of the surface ECG signals with the IEGM signal may improve the evaluation of the signals.

In one example of the method, the surface ECG is recorded with 64 channels. In the context of the present disclosure and for all embodiments described herein, fewer or more channels for the recording are also possible, such as e.g. 6, 32, 128, etc. The use of a greater number of channels enhances the precision of the signal analysis and involves greater computational complexity.

ICA analysis (ICA=Independent Component Analysis) is a known analysis method in signals processing that can reconstruct statistically independent signal sources that are present in a linear mixed signal. See, for example, the publication entitled “Independent Component Analysis” by A. Hyvärinen, J. Karhunen, and E. Oja, John Wiley & Sons, Inc. from 2001, in which the manner in which ICA functions is explained using its basic mathematical algorithms.

In this case, ICA analysis is performed on the data set of a multichannel surface ECG, preferably having 64 channels, and the data set is processed in order to determine individually a plurality of action potentials of the heart that represent signal sources, including their sum and their position. In the context of the present disclosure, the term “sum” of the action potential shall be construed to be the time-dependent curve of the specific potential.

ICA analysis may be used since the measurement points (electrodes/channels of the ECG) are arranged spatially distributed. They are attached to predetermined locations on the surface of the patient's body. Moreover, it is assumed that the number of measurement points (channels) is greater than the number of the statistically independent signal sources (here: action potentials in the heart). In addition, in this case the detected mixed signals (channels of the surface ECG) are a linear combination of the native signal sources (action potentials of the heart) or are a sufficiently precise linear combination for the subsequent calculations. Further, the action potentials do not have a Gaussian distribution and are statistically independent.

It is furthermore provided to record at least one intracardiac (preferably unipolar) ECG signal (hereinafter referred to as IEGM signal, for short) that is detected by means of at least one electrode arranged inside the heart. For this, a catheter, the electrode being arranged at the tip thereof, is advanced via the venous system into the patient's heart and is guided transseptally into the left atrium. The catheter is guided along on the inner wall of the chamber in order to measure the electrical potential at points on the inner wall by means of the electrode. The at least one electrode may also be used for ablation.

In one embodiment, the ICA analysis may also be applied to the IEGM signal. The IEGM signal is a composite signal that consists of individual signals generated by various electrical action potentials. ICA permits the individual signals to be extracted and isolated from the IEGM. To this end, the calculating steps typical for ICA are applied to the IEGM signal.

The IEGM signal is now compared to the plurality of action potentials determined from the surface ECG data set by means of ICA analysis, or to combinations of these action potentials, and thus analyzed combined. Based on this comparison, the sum and/or the position of at least one of the plurality of action potentials in the heart is corrected. Using this comparison, an action potential from the surface ECG evaluated by means of ICA analysis or a combination of such action potentials may be associated with the action potential found by means of IEGM signal. Since with the detected IEGM signal, the site of the associated action potential is known with much greater precision, the IEGM signal permits correction of the plurality of action potentials determined by means of ICA analysis.

This solution has the advantage that regions disposed even deeper in the cardiac muscle may be examined. By combining the signal of the surface ECG with the IEGM signal, it is not just surface signal paths that are detected. Action potentials from areas of the cardiac muscles disposed even deeper are added to the signal of the surface ECG and are worked out by being broken down into signal sources by means of the ICA analysis. If, for example, following an ablation only the preferably unipolar IEGM signal is significantly changed for the site in question or disappears, it is very probable that there is another conducting path in a deep-lying region of the myocardium and that this deep-lying region still has to be treated by means of ablation. The ablation may be assessed more precisely using the method.

In another exemplary embodiment, the comparison in step d) is performed by means of a correlation calculation. This makes possible a particularly more rapid, and simple to implement, comparison of the data. For defining the correlation in signals processing, see also: Daniel Ch. Von Grünigen—“Digitale Signalverarbeitung [Digital Signals Processing],” Carl Hanser Verlag GmbH & Co. KG, 2nd Edition, 2002, in particular page 51. For the correlation calculation from a more general perspective of statistics, see also: A. Papoulis—“Probability, Random Variables, and Stochastic Processes,” McGraw Hill, 3rd edition, 1991.

In another exemplary embodiment, the sum and position of the plurality of action potentials determined according to steps a) through d) are preferably illustrated by means of a two-dimensional or three-dimensional model of the heart in a two-dimensional or three-dimensional map. In this way the distribution of the action potentials may be evaluated particularly effectively.

In this method, voltage maps of the heart are calculated based on electrical potentials measured in and on the heart, i.e., the measured ECG signals. In connection with the present disclosure, ECG components that were determined according to the method may be used for calculating a voltage map.

It is furthermore advantageous when, based on the plurality of action potentials determined according to steps a) through d), a rotor and/or an ectopic action potential is automatically identified. Such a pathological action potential may be, e.g., depicted highlighted appropriately, for instance in a two-dimensional or three-dimensional map. In addition or alternatively, other fixed anatomical points and regions may be identified automatically, such as sinus nodes, atrial muscle, AV nodes, bundle branches, ventricular muscle, and may particularly preferably also, e.g., be depicted highlighted in a two-dimensional or three-dimensional map. For example, individual signal components may have specific properties for identifying an abnormal stimulus transmission or stimulus origin. For example, individual signal components are recorded as a function of time and location during the cardiac cycle and analyzed with known data over the normal conduction system of the heart. Such an analysis may represent from a comparison of certain characteristic features of the signal components with the known data, such as, for example, a comparison of threshold values for the signal amplitudes. An analysis from a morphology comparison of the signal components to a known signal pattern may also represent. The known signal pattern stands for a normal cardiac conduction system and may be generated specific to the patient, e.g. in that signals are recorded during the normal intrinsic heart activity and averaged to form a pattern signal. The signal pattern may also represent a previously stored pattern. If the analysis indicates that the signal components deviate from properties of a normal cardiac conduction system, rotors or ectopic action potentials could be the reason. Such signal components may be graphically highlighted when a map is created and displayed. If there is a rotor, the temporal and local analysis of the amplitudes of the corresponding signal components will have a circular signal path. Such components may then be highlighted, e.g. methods for morphological image processing may be used for this.

In one exemplary embodiment, the sum and position of a plurality of action potentials in the heart are determined with the steps a) through d), each in a first time period and in a second time period, the second time period being different from the first time period. The first time period is prior to an ablation, for example, and the second time period is after an ablation. In the next step, the change in the sum and/or position for some of the action potentials of the second time period relative to the first time period is determined. The change may be determined by means of a data processing device (e.g. personal computer, notebook, tablet, or smartphone). The data processing device may have a processor and a storage element.

In one exemplary embodiment, in step c) the detected multichannel surface ECG is broken down by ICA into a plurality of components, of which some represent action potentials of the heart or combinations thereof. Other components correspond, e.g. to artefacts of muscle movements, respiration movements, or signal interferences by external sources such as, e.g. humming from electrical sources. Parallel to the surface ECG, an IEGM signal is also detected at the ablation site. At the ablation site the intent is to interrupt the heart's signal forwarding or to suppress an undesired action potential. The IEGM signal detected simultaneously with the surface ECG is then compared to the ICA components, e.g. using cross-correlation, and the component that yields the greatest correlation factor is allocated to the site at which the cardiac tissue must be ablated. the ablation is then performed and steps a through d are repeated. If the signal is then measured again at the ablated site and the ICA component that was allocated to the same site in the first step using cross-correlation is still present, then the ablation was most likely not successful. It is possible to use the method not only for one IEGM measurement point, but for a plurality of IEGM measurement points, prior to and following the ablation.

When there is a change in the sum of an action potential by more than a prespecified threshold between the first time period and the second time period, it is particularly preferable when there is a corresponding acoustic and/or visual and/or tactile indication. In this case, following an ablation it is assumed that the ablation at the site in question was successful.

An object presented in the foregoing is furthermore attained using a computer program product for determining the magnitude and arrangement of a plurality of action potentials in the heart with program code means for executing a computer program following its implementation in a data processing device, the program code means being provided for executing the method described in the foregoing following implementation in the data processing device. The computer program product possesses the advantages explained above with respect to the method.

An object presented in the foregoing is furthermore attained using a device that comprises the following elements:

a receiving device for receiving a data set from a surface ECG signal that was recorded synchronously with a plurality of channels, and data from at least one IEGM signal of the associated heart recorded during the same time period, and,

a data processing device that is designed
to process the data set of the surface ECG signal by means of ICA analysis and to determine the sum and position of a plurality of action potentials in the heart based on the ICA analysis, and,
to compare the data from the at least one IEGM signal to the plurality of action potentials and to correct the sum and/or position of at least one action potential of the plurality of action potentials in the heart based on this comparison.

A microprocessor, such as, for example, a personal computer with multicore processor is used for the data processing device.

In one embodiment of the device, the surface ECG may be recorded with 64 channels. However, ECG recordings having fewer or greater than 64 channels are also possible.

Analogous to the method, the device is designed such that the success of an ablation may be assessed with greater precision because deeper regions of the myocardium may be included in the analysis, as well.

In one preferred exemplary embodiment, the data processing device is designed for performing the comparison by means of correlation calculation. As was stated in the foregoing, a rapid comparison of the data is possible by means of the correlation calculation.

In another exemplary embodiment, the device furthermore has a display device (for example in the form of a screen having a suitable resolution, e.g. 1920×1080 pixels) that is designed to represent the sum and the position of the determined plurality of action potentials preferably by means of a two-dimensional or three-dimensional model of the heart in a two-dimensional or three-dimensional map.

In another exemplary embodiment, the data processing device is designed to identify automatically a rotor and/or an ectopic action potential based on the determined plurality of action potentials. This simplifies the analysis of the data by the person providing treatment.

For evaluating the effectiveness of an ablation treatment, it is in particular advantageous when the data processing device is designed to determine the sum and position of a plurality of action potentials in the heart, both in a first time period (e.g. prior to the ablation) and in a second time period (e.g. following the ablation), the second time period being different from the first time period, and to determine the change in the sum and/or position in the plurality of action potentials in the second time period from those in the first time period.

The interpretation of the measurement results and the evaluation of the ablation is further simplified if the data processing device is designed to produce an indication signal that outputs an acoustic and/or visual and/or tactile indication on a display device (e.g. a screen, loudspeaker, or the like) when there is a change in the sum of an action potential between the first time period and the second time period by more than a predetermined threshold.

An object presented in the foregoing is also attained for the same reasons using a system comprising a device described in the foregoing and a recording device for a surface ECG signal synchronously with a plurality of channels, for example with 32 or 64 channels, and/or a recording device for at least one IEGM signal, device and recording device for a surface ECG signal and/or the recording device for at least one IEGM signal being connected such that the recording device for a surface ECG signal transmits the surface ECG signal to the device and the recording device for at least one IEGM signal transmits the at least one determined IEGM signal to the device.

Further features, aspects, objects, advantages, and possible applications of the present invention will become apparent from a study of the exemplary embodiments and examples described below, in combination with the figures, and the appended claims.

### DESCRIPTION OF EMBODIMENTS

The present invention is explained in the following using exemplary embodiments and referencing the figures. All described and/or graphically depicted features, alone or in any combination, form the subject matter of the present invention, regardless of their inclusion in the claims and regardless of references back to them.

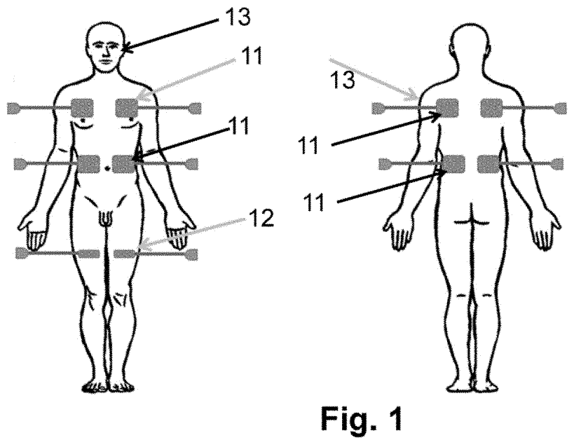

FIG. 1 illustrates the arrangement of the electrodes for the recording device on the body of the patient;

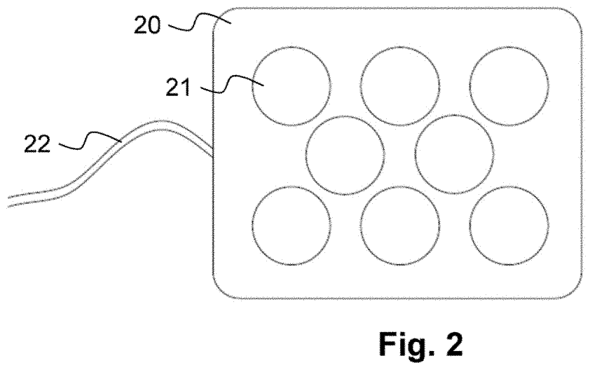

FIG. 2 is an enlarged schematic representation of an 8× electrode;

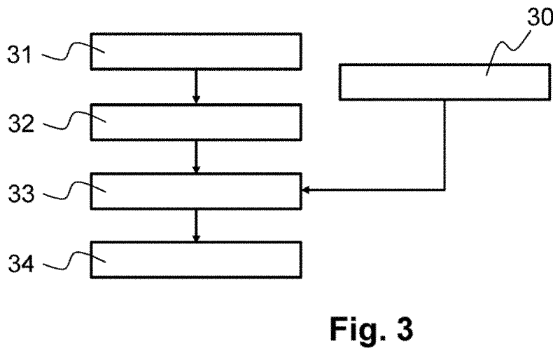

FIG. 3 illustrates one exemplary embodiment of a method; and

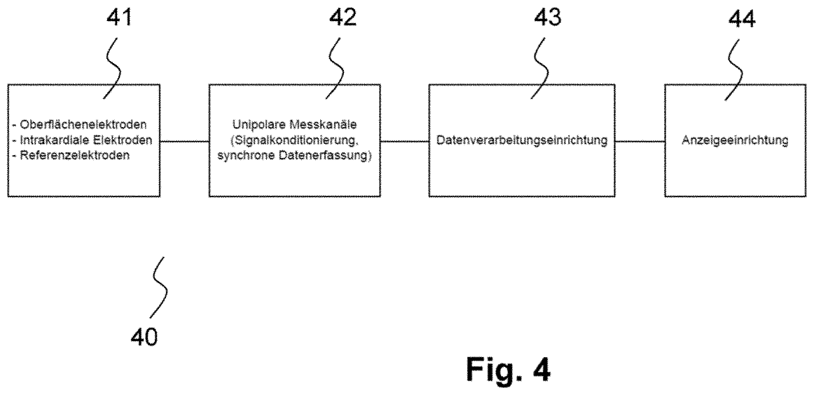

FIG. 4 is a schematic arrangement of the system for measuring ablation success.

### DETAILED DESCRIPTION

The system comprises a data processing device (for example, a processor of a computer), a catheter having an electrode or a plurality of electrodes at the distal catheter tip, and a device for taking a surface ECG. The surface ECG is detected in a unipolar manner against a reference electrode by means of special electrodes via a plurality of channels (see step 31 in FIG. 3). One example for the arrangement of 8× electrodes 11 and reference electrodes 12 on the body of the patient 13 is illustrated in FIG. 1. The reference electrodes for surface ECG and for unipolar IEGM are attached, e.g. frontally on the legs (as shown in FIG. 1). In one preferred embodiment, 8 patch electrodes are used, each having 8 individual electrodes, so that overall the ECG is measured via 64 channels. The channels for surface ECG and IEGM channels are sampled synchronously, e.g. with 24 bit resolution.

FIG. 2 is an enlarged illustration of the 8× electrode 11 from FIG. 1. Individual electrodes 21 that are arranged on a surface 20 may be seen, the electrodes being connected to the ECG measurement system via lead 22. The 8× electrode is preferably a patch electrode for recording ECGs that is adhesively applied to the patient's chest. A smaller or greater number of electrodes is also possible. The electrodes 21 are arranged offset to one another so that the largest possible surface area 20 is provided with electrodes. In this way, an electrical map of the surface potentials, with high spatial resolution, may be generated in the ECG measurement. However, other arrangements of the electrodes 21 are also suitable provided they are arranged such that via them cardiac signals can be recorded in a suitable manner.

The surface ECG data set thus found is then forwarded to the data processing device and processed there by means of ICA analysis (see step 32 in FIG. 3). ICA algorithms such as infomax, fastICA, Molgedy-Schuster ICA, or JADE are used for this, for example (see also A. Hyvärinen, J. Karhunen, and E. Oja, “Independent Component Analysis,” John Wiley & Sons, Inc., 2001, for a number of fastICA variants and for infomax-based ICA; Jean-Francois Cardoso “High-Order Contrasts for Independent Component Analysis,” Neural Computation 11, 157-192 (1999) for JADE; Molgedey L, Schuster H. “Separation of independent signals using time-delayed correlations,” Phys Rev Lett. 1994; 72:3634-3637 for Molgedey-Schuster ICA). The sum and position of a plurality of action potentials in the heart are determined by means of ICA analysis of the data set from the surface ECG signal. The maximum number of action potentials in this exemplary embodiment is 64.

Preferably at the same time the surface ECGs are taken, an IEGM signal is recorded by means of a catheter previously arranged in the heart, in particular by means of its electrode or electrodes (step 30 in FIG. 3). When a plurality of electrodes are used, the IEGM signal may be a multi-channel signal. Now the IEGM signal is preferably compared, by means of cross correlation, to the plurality of signal components, representing different signal sources, that were determined by the surface ECG and the subsequent ICA analysis (step 33 in FIG. 3). There may then be a correction of the action potentials, in terms of their sum and position, determined by means of the surface ECG (step 34 in FIG. 3.) If there is a temporal offset between the surface ECG signal and the IEGM signal, it may be ignored. A cross-correlation factor is preferably determined during the cross-correlation.

In order to observe the progress of the treatment during an ablation, the method explained in the foregoing is conducted in a first time period prior to an ablation (e.g. a few seconds to a few minutes prior to an ablation, depending on the cardiac cycle and the repetition period of the sought atypical cardiac signal) and a plurality of action potentials, including sum and position, are determined and, where necessary, corrected based on the IEGM signal. In addition, following the ablation, a second plurality of action potentials, including sum and position, are determined by means of the above method. The plurality of the determined action potentials prior to the ablation is then compared to the plurality of the determined action potentials following the ablation. Following the ablation, if there is still an action potential that corresponds to the ablation site, the lesion created by the ablation very probably did not completely reach the signal path or the ectopic source did not completely reach the depth of the myocardium. If this is the case, the ablation must be continued.

In addition, using the ICA analysis it is possible to undertake non-invasive reconstruction of the local action potentials of rotor, ectopic regions, sinus nodes, atrial muscle, AF nodes, HIS bundle, bundle branches, and ventricular muscle. In doing so the regular cardiac signals are recognized by registration algorithms. A data base having regular cardiac signals is required for this. The components may provide information about potential pathological changes in cardiac activity. Such an analysis may also take place without the use of a catheter (e.g., without being combined with an IEGM signal).

FIG. 4 depicts a schematic arrangement 40 of the essential components of the system for measuring the ablation outcome during an ablation in the left ventricle. The surface and intracardiac electrodes on the patient 41, including the reference electrodes, permit measurement of the surface ECG and the IEGM and are connected to multiple unipolar measurement channels 42; data are detected synchronously and signal processing is performed. The detected data are forwarded to data processing device 43, where they are evaluated and assessed by means of ICA and the correlation calculation, the assessment outcome in the context of this disclosure providing information on the outcome of the AF ablation. For this, at least one data set of the surface ECG signal is processed by the data processing unit by means of ICA analysis, the sum and position of a plurality of action potentials in the heart being determined based on the ICA analysis. Moreover, the data of the at least one IEGM signal is compared to a plurality of action potentials. Based on the comparison, the sum and/or position of at least one action potential from the plurality of action potentials in the heart is corrected. The data processing device 43 is connected to a display device 44 on which the assessment result regarding the outcome of the AF ablation may be displayed for a user. Graphic concepts, such as the display of a color scale or percentage scale, are suitable as means for indicating the ablation outcome on the display device 44. In addition, a calculated structure of the chambers of the heart (“map”) may be depicted on the display, together with the position of the catheter and the indication of ablation outcome at the specific site undergoing ablation.

It will be apparent to those skilled in the art that numerous modifications and variations of the described examples and embodiments are possible in light of the above teachings of the disclosure. The disclosed examples and embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A method for determining a plurality of action potentials in the heart having the following steps:
a) Recording surface ECG signals synchronously with a plurality of channels,
b) Recording at least one IEGM signal,
c) Processing the surface ECG signal by means of ICA analysis and determining the sum and position of a plurality of action potentials in the heart based on the ICA analysis,
d) Comparing the at least one IEGM signal to the plurality of action potentials and correcting the sum and/or the position of at least one of the plurality of action potentials in the heart based on this comparison.

2. The method according to claim 1, wherein the surface ECG is recorded synchronously with a plurality of channels, preferably 32 or 64 channels.

3. The method according to claim 1, wherein the comparison in step d) is performed by means of correlation calculation.

4. The method according to claim 1, wherein the sum and the position of the plurality of action potentials determined according to steps a) through d) are preferably illustrated by means of a two-dimensional or three-dimensional model of the heart in a two-dimensional or three-dimensional map.

5. The method according to claim 1, wherein a rotor and/or an ectopic action potential is identified based on the plurality of action potentials determined according to steps a) through d).

6. The method according to claim 1, wherein the sum and the position of a plurality of action potentials in the heart are determined with steps a) through d), each in a first time period and in a second time period, the second time period being different from the first time period, and in that the change in the sum and/or position of the plurality of action potentials of the second time period is determined relative to the first time period.

7. The method according to claim 6, wherein when there is a change in the sum of an action potential by more than a prespecified threshold between the first time period and the second time period, there is a corresponding acoustic and/or visual and/or tactile indication.

8. A computer program product for determining a plurality of action potentials in the heart with program code means for executing a computer program following its implementation in a data processing device, characterized in that the program code means are provided for executing the method according to claim 1 following implementation in the data processing device.

9. A device for determining a plurality of action potentials in the heart, comprising
a receiving device for receiving a data set from a surface ECG signal that was recorded synchronously with a plurality of channels, preferably with 32 or 64 channels, and data from at least one IEGM signal of the associated heart recorded during the same time period, and,
a data processing device that is designed
to process the data set of the surface ECG signal by means of ICA analysis and to determine the sum and position of a plurality of action potentials in the heart based on the ICA analysis, and
to compare the data from the at least one IEGM signal to the plurality of action potentials and to correct the sum and/or position of at least one action potential of the plurality of action potentials in the heart based on this comparison.

10. The device according to claim 9, wherein the data processing device is designed for performing the comparison by means of correlation calculation.

11. The device according to claim 9, wherein the device furthermore has a display device that is designed to represent the sum and position of the determined plurality of action potentials preferably by means of a two-dimensional or three-dimensional model of the heart in a two-dimensional or three-dimensional map.

12. The device according to claim 9, wherein the data processing device is designed to identify automatically a rotor and/or an ectopic action potential based on the determined plurality of action potentials.

13. The device according to claim 9, wherein the data processing device is designed to determine the sum and position of a plurality of action potentials in the heart, both in a first time period and in a second time period, the second time period being different from the first time period, and to determine the change in the sum and/or position in the plurality of action potentials in the second time period relative to those in the first time period.

14. The device according to claim 13, wherein the data processing device is designed to produce an indication signal that outputs an acoustic and/or visual and/or tactile indication on a display device when there is a change in the sum of an action potential between the first time period and the second time period by more than a predetermined threshold.

15. A system comprising a device according to claim 9 and a recording device for a surface ECG signal synchronously with a plurality of channels, preferably with 32 or 64 channels, and/or a recording device for at least one IEGM signal, device and recording device for a surface ECG signal and/or the recording device for at least one IEGM signal being connected such that the recording device for a surface ECG signal transmits the determined surface ECG signal to the device and the recording device for at least one IEGM signal transmits the at least one determined IEGM signal to the device.
